(19) United States
(12) Plant Patent Application Publication
(10) Pub. No.: US 2003/0177553 P1
(43) Pub. Date: Sep. 18, 2003

(54) Hybrid tea rose plant named'Korsered'

(75) Inventor: Tim-Hermann Kordes, (US)

Correspondence Address: Russell D. Orkin, 700 Koppers Building, 436 Seventh Avenue, Pittsburgh, PA, 15219-1818, US

(73) Assignee: W. Kordes Sohne Rosenschulen GMBH & Co. KG

(21) Appl. No.: 10/100,424

(22) Filed: Mar. 18, 2002

(51) Int. Cl. A01H 5/00

(52) U.S. Cl. PLT/139

(57) ABSTRACT

This invention relates to a new and distinct variety of hybrid tea rose plant named ‘KORsered’ which has attractive dark red colored flowers and attractive dark green and glossy foliage. This new variety is characterized by having a high production of long stems with a long vase life, vigorous and upright growth, and good tolerance to powdery mildew. The variety successfully propagates from softwood cuttings and is well suitable for year round production of cut flowers in commercial glasshouses as a flowering cut rose.

### BACKGROUND OF THE INVENTION

The present invention constitutes a new and distinct variety of hybrid tea rose plant named ‘KORsered’ which was developed by artificially pollinating an unnamed, unpatented seedling with an unnamed, unpatented seedling. The two parents were crossed in the summer of 1999 and the resulting seed was sown in December 1999 in a controlled greenhouse environment. The objectives of the hybridization of this rose variety for commercial greenhouse culture was to create a new and distinct variety with:

1. Uniform, large buds that are well turbinated and flowers with good vase-life.

2. Novel flower color.

3. High cut flower production, with medium to long stems.

4. Vigorous, upright growth.

5. Suitability for production from softwood cuttings.

6. Good tolerance to powdery mildew.

7. Suitability for production of cut flowers under greenhouse growing conditions.

This combination of qualities was not present in previously available commercial cultivars of this type and distinguish ‘KORsered’ from other varieties known to the inventor.

The seeds from hybridization were planted in a controlled environment and evaluations were conducted on the resulting plants. ‘KORsered’ was selected in a development program in Klein Offenseth, Sparrieshoop, Germany.

Asexual reproduction of ‘KORsered’ by cuttings was first done on Mar. 31, 2002 in Germany and later in California. The reproductions by cuttings done in controlled greenhouse environment have confirmed that distinguishing features of the new cultivar come true, remain stable and are retained through successive propagations. ‘KORsered’ is a highly productive hybrid tea rose plant with a vase life greater than 12 days. Under conventional greenhouse conditions in Germany (64° C. nights, 26° C. days), ‘KORsered’ will produce 250 stems averaging 55 cm to 65 cm long.

The new rose is distinguished from its seed parent by the following combination of characteristics:

1. ‘KORsered’ has big double flowers, while the unnamed seedling has big single flowers.

2. ‘KORsered’ has dark red colored petals, while the unnamed seedling has dark orange-red petals.

The new variety is distinguished from its pollen parent by the following combination of characteristics:

1. ‘KORsered’ has bigger flowers and foliage as compared to the unnamed seedling.

2. ‘KORsered’ has dark red colored petals, while the unnamed seedling has dark pink petals.

### DESCRIPTION OF THE DRAWINGS

The accompanying color illustrations show as true as possible in color photographs of this type, the typical characteristics of the buds, flowers, leaves, stems of ‘KORsered’. Specifically illustrated as follows:

In photo sheet #1:

FIG. 1 illustrates a young shoot;

FIG. 2 illustrates a bud before opening of the sepals;

FIG. 3 illustrates a bud at the opening of the sepals;

FIG. 4 illustrates a bud at the opening of the petals;

FIG. 5 illustrates a flower during course of opening;

FIG. 6 illustrates an open flower, plan obverse view;

FIG. 7 illustrates an open flower, plan reverse view;

FIG. 8 illustrates a fully open flower, plan obverse view; and

FIG. 9 illustrates a fully open flower, plan reverse view.

In photo sheet #2:

FIG. 10 illustrates a receptacle showing stamens and pistils;

FIG. 11 illustrates a receptacle showing pistils (stamens removed);

FIG. 12 illustrates flower petals detached, outer surface;

FIG. 13 illustrates flower petals detached, inner surface;

FIG. 14 illustrates a bare stem exhibiting thorns and flower attachment;

FIG. 15 illustrates 3 leaflets, upper side;

FIG. 16 illustrates 3 leaflets, under side;

FIG. 17 illustrates 5 leaflets, upper side; and

FIG. 18 illustrates 5 leaflets, under side.

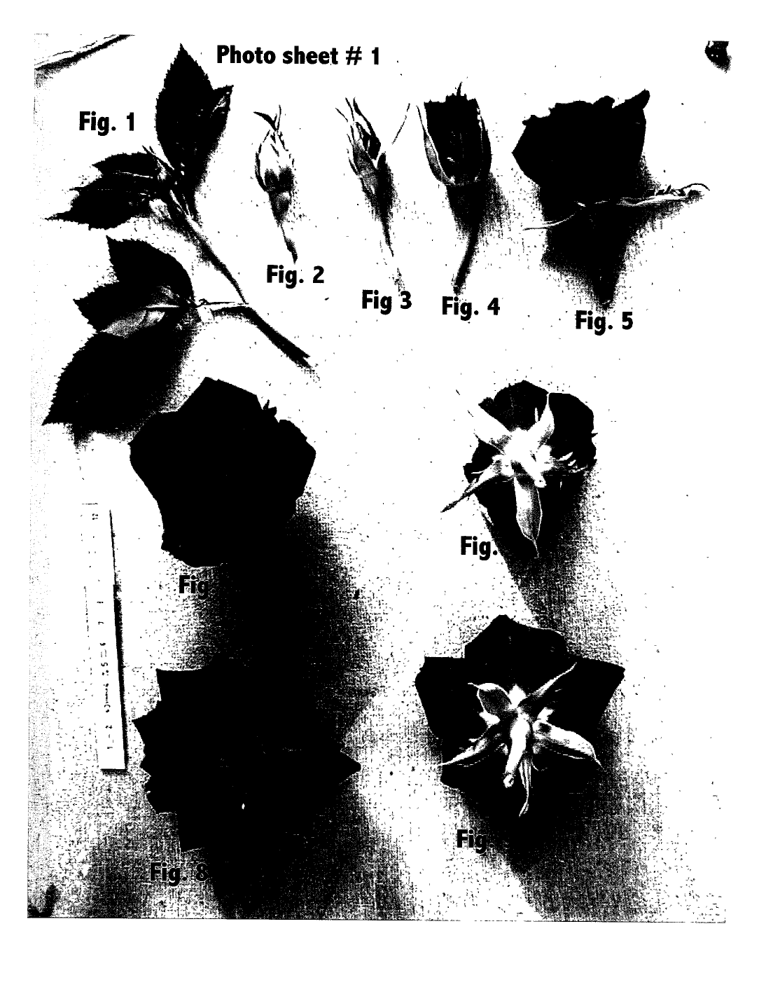

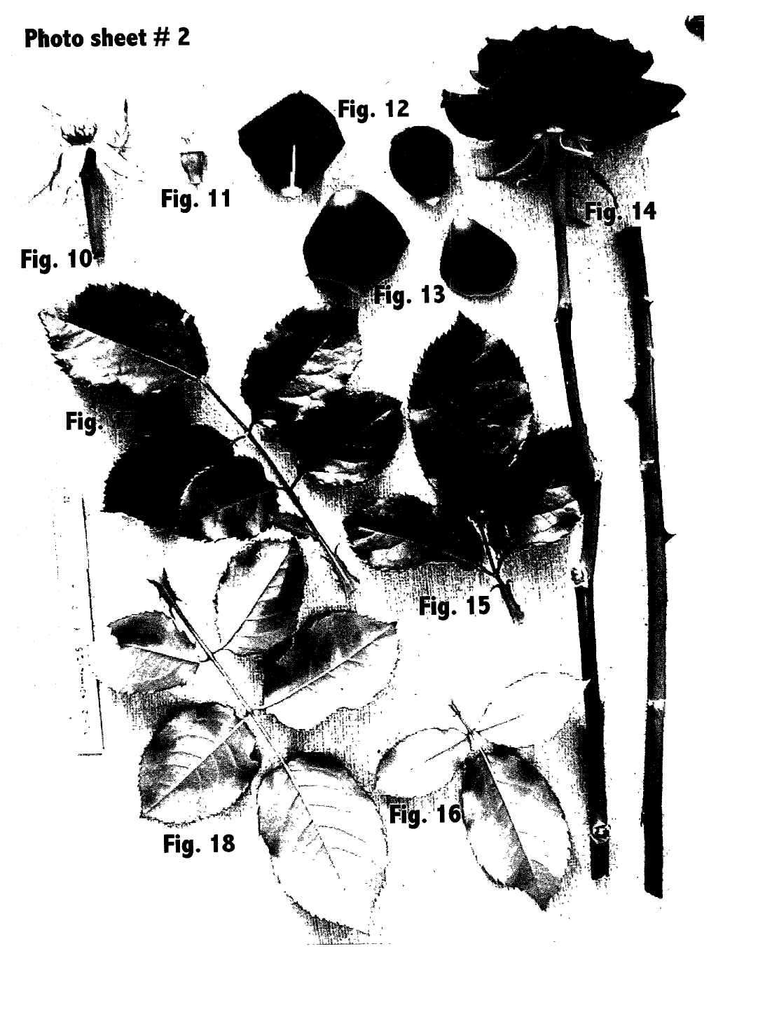

### DESCRIPTION OF THE PLANT

The following is a detailed description of ‘KORsered’, as observed in its growth in greenhouses in Klein Offenseth-Sparrieshoop, Germany, and glasshouses in California. The age of the observed plant was 11 to 13 months after propagation, produced as a cut rose plant. The cultivation at both locations was based on 18 to 26° C. temperatures. Color references are made using The Royal Horticultural Society (London, England) Colour Chart, 1995, except where common terms of color are used. For a comparison, the nearest existing rose variety is ‘JACkisca’, U.S. Plant Pat. No. 11,776.

Table 1 details several physical characteristics of ‘KORsered’ and ‘JACkisca’.

TABLE 1

| | KORsered | JACkisca |
|---|---|---|
| Petal color, upper surface | 45B | 53B |
| Petal color, reverse surface | 45B | 53D |
| Petal count | 35-40 | 40-45 |

### THE PLANT

Classification:

Botanical.—Rosa hybrida.

Commercial.—Hybrid tea rose.

Growth habit: Very vigorous. Grows strong, upright to bushy. Production time for floral stems is generally 6-8 weeks depending on average temperature, light level, and cultural practices.

Height: Plants which were pruned at a height of 0.5 meter, produced floral stems of 70-95 cm long with a range of 60-115 cm.

Stem:

Color.—Young wood: 144A. Older wood: 147A.

Thorns:

Incidence.—Low number of thorns.

Size.—8-10 mm.

Color.—178A.

Shape.—Concave.

Surface:

Young wood.—Smooth, with a few prickles colored 178A.

Older wood.—Smooth.

Stem diameter: 8-10 mm.

Internode length: 50-65 mm.

Number of internodes: 12-14.

### FOLIAGE

Arrangement: Alternate, compound with 3-7 leaflets per leaf, generally symmetrical, abundant, and flat in aspect. Stipules at petiole base.

Quantity of leaves: 12-14 per lateral branch.

Leaf size:

Length.—135-170 mm.

Width.—130-140 mm.

Petioles:

Color.—145A.

Margin.—Entire, with few prickles colored 178A.

Length.—5-20 mm.

Diameter.—About 1-2 mm.

Stipules:

Size.—15-20 mm.

Surface.—Smooth.

Color.—137D with intonations of 178A.

Margin.—Serrated, single.

Rachis:

Color.—145B with intonations of 178A.

Margin.—Having few prickles colored 178A.

Length.—30-50 mm.

Leaflets:

Edge.—Serrated.

Serration.—Single.

Shape.—Ovate with acute apex and obtuse base.

Texture.—Smooth.

Appearance.—Glossy.

Size.—Length: 50-80 mm. Width: 30-50 mm.

Color.—Young foliage: Upper surface: 144A with intonations of 178A. Lower surface: 146A with intonations of 178A. Mature foliage: Upper surface: 137A. Lower surface: 191A with intonations of 178A.

### BUD

Size:

Height.—35-45 mm.

Width.—30-35 mm.

Shape: Cupped.

Color: 45B at one-quarter open.

### INFLORESCENCE

Flowering characteristics: Recurrent, generally 1 bud per flowering stem.

Duration: On the plant, flowers last 16-18 days. When cut and placed in a vase, flowers last 12-14 days.

Fragrance: None.

Size: 90-100 mm in diameter.

Shape of flower when viewed from side:

Upon opening.—Cupped.

Open flower.—Cupped.

Peduncle:

Color.—144A.

Texture.—Smooth, with pubescence and prickles.

Length.—65-70 mm.

Angle.—Upright.

Receptacle:

Surface.—Smooth, glabrous.

Shape.—Funnel-shaped.

Size.—Height: 10-12 mm. Width: 10-12 mm.

Color.—144A.

Sepals:

Quantity.—5.

Shape.—Narrowly ovate with acute tip.

Texture.—Leathery.

Margin.—Foliaceous appendages on 3 of the 5 sepals.

Appearance.—Dull.

Color.—Upper surface: 144A. Lower surface: 144A.

Petals:

Color.—Upon opening: Upper surface: 45B. Lower surface: 45B. After opening: Upper surface: 46C. Lower surface: 45C.

General tonality on open flower.—46C on third day, 45C afterwards.

Basal petal spots.—Size: 4-5 mm. Color: 155A.

Reflex.—Outermost petals reflex backwards at opening. When fully open all petals reflex backwards.

Texture.—Smooth.

Margin.—Entire.

Number.—Approximately 35-40 on average per flower.

Size.—Length: 40 mm. Width: 40 mm.

Shape.—Outer petals: Round. Inner petals: Ovate.

Petaloids: Usually none.

### REPRODUCTIVE ORGANS

Stamen: Approximately 125-130 on average per flower.

Pollen.—Color: 163A. Amount: Average.

Anthers.—Size: 3-4 mm. Color: 163A. Shape: Oblong.

Filaments.—Size: 10-12 mm. Color: 46A.

Pistils: Approximately 80-85 on average per flower.

Stigmas.—Location: Superior to anthers. Color: 155C.

Styles.—Color: 46A. Length: 3-4 mm.

Aptitude to bear fruit: Poor.

Resistance to diseases: Above average resistance to mildew and Botrytis under normal growing conditions in Klein Offenseth-Sparrieshoop, Germany and California.

Productivity: Commonly produces approximately 200-300 flowers/m²·year under greenhouse conditions.

Hips/seeds: Unknown, the plant has not been grown to the stage of seed development due to the fact that the variety is developed for use as a flowering cut rose only.

Winter hardiness and drought/heat tolerance: Due to the fact that this variety is a flowering cut rose plant developed for greenhouse cultivation only, the plant has not been tested for winter hardiness or drought/heat tolerance.

I claim:

1. The new and distinct variety of hybrid tea rose plant named ‘KORsered’ as described and illustrated.
